(19) United States
(12) Patent Application Publication
(10) Pub. No.: US 2013/0004490 A1
(43) Pub. Date: Jan. 3, 2013

(54) DELIVERY OF TRANSTHYRETIN ACROSS THE BLOOD-BRAIN BARRIER AS A TREATMENT FOR ALZHEIMER'S DISEASE

(75) Inventors: Juan Jose Marugan, Gaithersburg, MD (US); Wei Zheng, Potomac, MD (US); Silvia Muro-Galindo, Silver Spring, MD (US)

(73) Assignees: The United States of America, as represented by the Secretary, Department; UNIVERSITY OF MARYLAND, COLLGE PARK, College Park, MD (US); of Health and Human Services, Bethesda, MD (US)

(21) Appl. No.: 13/515,050

(22) PCT Filed: Dec. 14, 2010
(86) PCT No.: PCT/US10/60205

§ 371 Date: Sep. 18, 2012

Related U.S. Application Data
(60) Provisional application No. 61/286,205, filed on Dec. 14, 2009.

(51) Int. Cl. A61K 39/395; C07K 16/18; A61K 39/44; C12N 15/62; A61P 25/28; C12N 1/21; C08G 63/91; C12P 21/02; C12N 15/63; C12N 1/19; C12N 5/10; A61K 38/17; C07K 19/00

(52) U.S. Cl. 424/134.1; 514/13.5; 530/387.3; 424/136.1; 536/23.4; 530/380; 525/54.1; 424/179.1; 530/391.1; 435/69.7; 435/320.1; 435/254.2; 435/419; 435/325; 435/348; 435/365; 435/358; 435/369; 435/252.33

(57) ABSTRACT

The invention provides a composition comprising transthyretin and an ICAM-1 targeting agent, wherein the transthyretin and ICAM-1 targeting agent are coupled together, as well as methods of preparing such compositions. The invention further provides a diabody capable of binding specifically to ICAM-1 and transthyretin. The invention also provides a method of use of such composition in the manufacture of a medicament for treating an amyloid-β related neurodegenerative disease, comprising administering to a subject a composition comprising transthyretin coupled to an ICAM-1 targeting agent in an amount effective to treat the neurodegenerative disease, wherein the composition is administered to the subject outside of the blood-brain barrier.

### CROSS-REFERENCE TO RELATED APPLICATIONS

This patent application claims the benefit of U.S. Provisional Patent Application No. 61/286,205, filed Dec. 14, 2009, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 14,486 Byte ASCII (Text) file named “707050SequenceListing_ST25.TXT,” created on Dec. 13, 2010.

### BACKGROUND OF THE INVENTION

Alzheimer's disease (AD) is a neurodegenerative condition leading to progressive cognitive deterioration and neuropsychiatric symptoms, as well as behavioral changes and inability to perform daily activities. AD is characterized by formation of extracellular plaques of amyloid β and intracellular tangles in areas of the brain providing cognitive function. Amyloid β also induces hyperphosphorylation of the microtuble associated protein tau, leading to formation of intracellular tangles. Conventional treatment of AD entails administration of cholinesterase inhibitors or memanatine, an N-methyl-D-aspartate receptor antagonist.

An obstacle in the development of treatments for AD and other diseases of the central nervous system (CNS) is the lack of safe and effective means to transport therapeutic agents from the blood to the brain.

Additional strategies are needed to provide effective therapy for neurodegenerative diseases, particularly AD.

### BRIEF SUMMARY OF THE INVENTION

In an aspect, the invention provides a composition comprising transthyretin and an intercellular adhesion molecule-1 (ICAM-1) targeting agent, wherein the transthyretin and ICAM-1 targeting agent are coupled together.

In another aspect, the invention provides a method of treating an amyloid-β related neurodegenerative disease, comprising administering to a subject a composition comprising transthyretin coupled to an ICAM-1 targeting agent in an amount effective to treat the neurodegenerative disease, wherein the composition is administered to the subject outside of the blood-brain barrier.

In yet another aspect, the invention provides methods of producing or preparing compositions comprising transthyretin and an ICAM-1 targeting agent, wherein the transthyretin and ICAM-1 targeting agent are coupled together.

In other aspects, the invention provides a diabody capable of binding specifically to transthyretin and ICAM-1, as well as methods of making and using such diabodies.

### BRIEF DESCRIPTION OF THE SEVERAL VIEWS OF THE DRAWING(S)

FIG. 1A depicts fluorescence microscopy results for FITC-labeled anti-ICAM polystyrene carriers in TNFα-activated human umbilical vein endothelial cells (HUVEC) (control), versus human brain microvascular endothelial cells (BMVEC), and human neuroblastoma SH-SY5Y cells. Surface bound carriers (Surface) and materials internalized through endocytosis (Internalized) are shown separately. Dashed lines mark the cell border, determined by phase-contrast microscopy. Magnification bar=10 μm.

FIG. 1B depicts fluorescence microscopy results for TNFα activated HUVEC incubated with anti-ICAM conjugates: streptavidin biotinylated anti-ICAM, prototype anti-ICAM polystyrene carriers, or biodegradable anti-ICAM poly-lactic co-glycolic acid (PLGA) carriers. Surface bound carriers (Surface) and materials internalized through endocytosis (Internalized) are shown separately. Dashed lines mark the cell border, determined by phase-contrast microscopy. Magnification bar=10 μm.

FIG. 2 depicts fluorescence microscopy results of brain tissue from mice injected with FITC-labeled polystyrene carriers coated with IgG as compared to anti-ICAM monoclonal antibody.

FIG. 3 depicts transmission electron microscopy results of brain tissue from mice injected with FITC-labeled polystyrene carriers coated anti-ICAM monoclonal antibody at eight hours after intravenous injection. Arrows indicate carriers which have been further transported across the blood-brain barriers into the Purkinje neuronal region of the cerebellum.

FIG. 4A depicts accumulation levels of an anti-ICAM polystyrene carrier and anti-Insulin Receptor (InsR) fusion protein by percent injected dose/gram. (Mean±SEM, n≧3 mice).

FIG. 4B depicts accumulation levels of anti-ICAM targeted polystyrene carriers administered via jugular vein versus carotid artery. Mean±SEM, n≧3 mice.

FIG. 5A depicts localization ratio data, which represents the percent injected dose/gram in brain divided by percent injected dose/gram in circulation in the blood for anti-ICAM monoclonal antibody or ICAM-1 affinity peptide γ3. Mean±SEM; n=2 assays or ≧3 mice. *p<0.05, **p<0.005, by student's t test.

FIG. 5B depicts relative binding of γ3 to human and mouse ICAM-1 (hICAM-1 and mICAM-1, respectively), as compared to positive controls (dashed line) and negative controls (dotted line).

FIG. 5C depicts relative binding of γ3 carriers to HUVEC cells alone (134±11 particles/cell) (unlabeled bar) or in the presence of anti-ICAM, γ3 peptide or γ3 scramble peptide, and compared to negative controls (dotted line). Mean±SEM; n=2 assays or ≧3 mice. **p<0.005, ***p<0.001, by student's t test.

FIG. 6A depicts accumulation levels of ICAM/¹²⁵I-TTR polystyrene carriers as compared to anti-ICAM/¹²⁵I-IgG polystyrene carriers as a control, calculated as % injected dose/gram of brain. Mean±SEM; n≧3 mice.

FIG. 6B depicts localization ratio of anti-ICAM/¹²⁵I-TTR polystyrene carriers compared to control ¹²⁵I-TTR, calculated as the Localization Ratio (% injected dose/gram of brain divided to % injected dose/gram in blood). Mean±SEM; n≧3 mice.

FIG. 7 provides a schematic illustration of an ICAM-1/TTR diabody and possible binding models thereof.

FIG. 8 provides a schematic illustration of an ICAM-1-TTR expression plasmid and the resulting chimeric protein.

### DETAILED DESCRIPTION OF THE INVENTION

The present invention relates to compositions and methods involving transthyretin, also known as prealbumin, a protein found in cerebrospinal fluid, coupled with an agent capable of targeting intercellular adhesion molecule-1 (ICAM-1), which is an immunoglobulin-family transmembrane glycoprotein expressed on endothelial cells in cerebrovascular areas, and in all vascularized organs throughout the body.

The transthyretin (TTR) can be any variant, analog, or homolog of TTR useful for therapeutic or research purposes in any human or non-human mammal. The TTR can have, for example, the sequence of human TTR, such as GenBank Accession Number NP_{—}000362.1.

TTR variants are polypeptides that differ in amino acid sequence from native TTR, but that retain at least one biological activity of native TTR, such as the ability to bind to amyloid β. A variant can be substantially identical to a native protein as described above. A sequence can also be a variant if the DNA encoding the sequence is capable of hybridizing under stringent conditions to the complement of DNA encoding a native TTR protein. Stringent conditions are conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions can be selected to be about 5-10° C. lower than the thermal melting point (Tₘ) for the specific sequence at a defined ionic strength pH. The Tₘ can be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions can also be achieved with the addition of destabilizing agents such as formamide. For selective or specific hybridization, a positive signal can be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

TTR homologs are polypeptides native to different species that retain biological activity (e.g., human and porcine insulin, human and salmon calcitonin, etc.) or intraspecies isomers of a polypeptide (protein “families” such as the cytochrome P450 family). Non-human TTR sequences are readily available to one of ordinary skill in the art via GenBank, at accession numbers such as NP_{—}038725.1 (Mus musculus); AAA86054.1 (Petaurus breviceps); AAh86946.1 (Rattus norvegicus); or XP_{—}419176.1 (Gallus gallus).

TTR analogs are polypeptides that differ in amino acid sequence from native TTR but retain at least one biological activity of a native TTR protein, as described above. These analogs can differ in amino acid sequence from TTR, e.g., by the insertion, or substitution of amino acids. Preferably, a substitution is conservative. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. See, e.g., Kyte et al., J. Mol. Biol. 157:105-132 (1982). The hydropathic index of an amino acid is based on a consideration of its hydrophobicity and charge, and include the following values: alanine (+1.8), arginine (−4.5), asparagine (−3.5), aspartate (−3.5), cysteine/cystine (+2.5), glycine (−0.4), glutamate (−3.5), glutamine (−3.5), histidine (−3.2), isoleucine (+4.5), leucine (+3.8), lysine (−3.9), methionine (+1.9), phenylalanine (+2.8), proline (−1.6), serine (−0.8), threonine (−0.7), tryptophan (−0.9), tyrosine (−1.3), and valine (+4.2). It is known in the art that amino acids of similar hydropathic indexes can be substituted and still retain protein function. Preferably, amino acids having hydropathic indexes of +/−2 are substituted.

The hydrophilicity of amino acids can also be used to reveal substitutions that would result in proteins retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity as described in U.S. Pat. No. 4,554,101. Hydrophilicity values for each of the common amino acids, as reported in U.S. Pat. No. 4,554,101, are: alanine (−0.5), arginine (+3.0), asparagine (+0.2), aspartate (+3.0.+−0.1), cysteine (−1.0), glycine (0), glutamate (+3.0.+−0.1), glutamine (+0.2), histidine (−0.5), isoleucine (−1.8), leucine (−1.8), lysine (+3.0), methionine (−1.3), phenylalanine (−2.5), proline (−0.5.+−0.1), serine (+0.3), threonine (−0.4), tryptophan (−3.4), tyrosine (−2.3), and valine (−1.5). Substitution of amino acids having similar hydrophilicity values can result in proteins retaining biological activity, for example immunogenicity, as is understood in the art. Preferably, substitutions are performed with amino acids having hydrophilicity values within +/−2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Additionally, computerized algorithms are available to assist in predicting amino acid sequence domains likely to be accessible to an aqueous solvent. These domains are known in the art to frequently be disposed towards the exterior of a protein, thereby potentially contributing to binding determinants, including antigenic determinants. Having the DNA sequence in hand, the preparation of such analogs is accomplished by methods well known in the art (e.g., site-directed) mutagenesis and other techniques.

TTR derivatives are proteins or peptides that differ from native TTR in ways other than primary structure (i.e., amino acid sequence). For example, polypeptides can exhibit glycosylation patterns due to expression in heterologous systems. The various polypeptides of the present invention, as described above, can be provided as discrete polypeptides or be linked, e.g., by covalent bonds, to other compounds. Thus, other TTR derivatives include, but are not limited to, fusion proteins having a covalently modified N or C-terminus, PEGylated polypeptides, polypeptides associated with lipid moieties, alkylated polypeptides, polypeptides linked via an amino acid side-chain functional group to other polypeptides or chemicals, and additional modifications as would be understood in the art. If these polypeptides retain at least one biological activity of a native TTR protein, then these polypeptides are TTR derivatives in the context of the invention.

Preferably, a variant, analog, homolog, or derivative of a native TTR or protein related to TTR has at least 60%, or more preferably, 70%, 75%, 80%, 85%, 90%, 95%, 99%, or 100% similarity to the native sequence. More preferably, a variant, analog, homolog, or derivative of a native TTR or protein related to TTR has at least 60%, or more preferably, 70%, 75%, 80%, 85%, 90%, 95%, 99%, or 100% sequence identity to the native sequence. A sequence is substantially identical if it is at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more amino acids.

Sequence identity and/or similarity can be determined using standard BLAST parameters or any other measure of sequence identity and/or similarity as known to one of ordinary skill in the art. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation.

Fragments of TTR, a protein related to TTR, or of a variant, analog, or homolog thereof that retain one or more activities of native TTR, and which have sufficient activity to protect injury as discussed herein, also are suitable TTR proteins for use in the context of the present invention. Biologically active fragments of TTR can be naturally occurring or non-naturally occurring, including recombinant fragments of a TTR or a protein related to TTR. Due to the high level of conservation of TTR proteins, it will be expected that one skilled in the art will identify biologically active sequences from the various proteins disclosed above. Preferably, a fragment for use in the present invention comprises a sequence of at least 5, such as at least 10, and more preferably at least 15 or at least 20, such as at least 30 or a least 50 contiguous amino acids of a native TTR, a protein related to TTR, or of a variant, analog, or homolog thereof. A fragment can include a sequence of substantially all of the native TTR, a protein related to TTR, or of a variant, analog, or homolog thereof or it can comprise less than substantially all of native TTR, a protein related to TTR, or of a variant, analog, or homolog thereof. Thus, for example, a fragment can comprise a sequence of up to about 147, such as up to about 140, or up to about 125, or up to about 100, or up to about 75 contiguous amino acids of a native TTR, a protein related to TTR, or of a variant, analog, or homolog thereof.

The ICAM-1 targeting agent can be any suitable agent capable of binding ICAM-1. In some embodiments, the ICAM-1-targeting agent is an anti-ICAM-1 antibody or fragment thereof, wherein the antibody or fragment is capable of binding ICAM-1 with specificity. One of ordinary skill in the art can easily prepare and isolate suitable antibodies to ICAM-1, which can be monoclonal or polyclonal, and which can also be humanized or fully human antibodies. Exemplary anti-ICAM-1 antibodies and fragments thereof are well known to one of ordinary skill in the art. See, e.g., WO91/106927; WO91/016928; Haug et al., Transplantation 55:766 (1993); Kavanaugh et al., Arthritis. Rheum. 37: 992 (1994); Wee et al., Transplant. Proc. 23: 279 (1991); and Argenbright et al., J. Leukocyte Biol. 49: 253 (1991). In other embodiments, the ICAM-1 targeting agent is γ3 (SEQ ID NO:7), a 17-mer polypeptide derived from fibrinogen which is an endogenous protein found in blood of human and non-human animals. In still other embodiments, the ICAM-1 targeting agent can be an ICAM-1 binding moiety identifiable by one of ordinary skill in the art such as, for example, an aptamer, a nucleic acid, a peptide, a peptidomimetic, a carbohydrate, a lipid, a vitamin, a toxin, a component of a microorganism, a hormone, a receptor ligand, any combination of these molecules, and/or any derivative thereof. Combinations of the foregoing ICAM-1 targeting agents can also be prepared and used.

In some embodiments, the transthyretin and ICAM-1 targeting agent are coupled as a complex or a fusion protein. Such a complex or fusion protein can be prepared using any method available to one of ordinary skill in the art. For example, TTR and the ICAM-1 targeting agent can be coupled using a method such as biotin-streptavidin conjugation, chemical conjugation, covalent coupling, antibody coupling, and direct expression (e.g., a chimeric protein). An exemplary chimeric ICAM-1-TTR nucleotide sequence for expression is provided at SEQ ID NO: 8. A sequence of the resulting expressed polypeptide is provided at SEQ ID NO: 9, while a post-cleavage product comprising γ3, a (Ser4Gly)2 spacer, and TTR is provided at SEQ ID NO: 10. Another exemplary chimeric ICAM-1-TTR nucleotide sequence is provided at SEQ ID NO: 11, with the resulting expressed polypeptide provided at SEQ ID NO: 12 and its post-cleavage product provided at SEQ ID NO: 13. In a preferred embodiment, TTR and the ICAM-1-targeting agent can be expressed as a chimeric protein from a plasmid inserted in E. coli or S2 insect cells. In other preferred embodiments, TTR and the ICAM-1-targeting agent can be expressed using a mammalian expression system such as Chinese Hamster Ovary (CHO). In still other embodiments, a diabody capable of binding specifically to both ICAM-1 and TTR (thus coupling ICAM-1 and TTR) can be prepared using methods known to one of ordinary skill in the art, such as described in Perisic et al., Structure 2(12): 1217 (1994). In some embodiments, the diabody can be administered in combination with exogenous TTR, which can be free or coupled to the diabody. Upon administration of such a diabody to a subject, the subject's endogenous TTR and/or the exogenous TTR can bind to the diabody, and thereby couple to the ICAM-1-targeting agent, at its TTR-specific binding region. In such embodiments, the diabody can target ICAM-1 or TTR in either order or simultaneously.

In other embodiments, the transthyretin and ICAM-1 targeting agent are coupled by a co-polymer nanocarrier. In some embodiments, particularly research embodiments, the co-polymer nanocarrier can be a polystyrene particle. In a more preferred embodiment, the co-polymer nanocarrier is a poly-lactic-co-glycolic acid (PLGA) co-polymer.

It will be understood by one of ordinary skill in the art that the ICAM-1-targeting agent, the TTR, and any carrier, such as a co-polymer nanocarrier, can be coupled in any combination, i.e., the ICAM-1-targeting agent can be coupled directly or through TTR to the carrier, and TTR can be coupled directly or through the ICAM-1-targeting agent to the carrier. It will be understood that one or multiple TTR molecules and multiple ICAM-1 targeting agent molecules can be coupled to the same carrier. The molecules can be combined in any suitable ratio. Contemplated relative percentages of TTR as compared to the ICAM-1 targeting agent can be determined according to mass ratio or molar ratio, and can range, for example, from about 0.1%-5%, 3%-7% TTR, about 5%-10% TTR, about 10%-20% TTR, about 25%-30% TTR, about 30%-40% TTR, about 35%-45% TTR, about 40%-50% TTR, about 45%-55% TTR, about 50%-75% TTR, about 60%-80% TTR, about 80%-90% TTR, about 85%-95% TTR, about 93%-97% TTR, and about 95%-99.9% TTR. It will be understood that in such composition the ICAM-1 targeting agent is present in the corresponding percentage, such that the sum of TTR and ICAM-1 targeting agent is about 100%. Intervening ratios are also contemplated and can be easily prepared by one of ordinary skill in the art. In a preferred embodiment, TTR and the ICAM-1 targeting agent are present in a ratio of about 5 percent TTR to about 95 percent ICAM-1 targeting agent.

In another aspect, the invention provides a method of treating an amyloid-β related disease, comprising administering to a patient a composition comprising transthyretin coupled to an ICAM-1 targeting agent in an amount effective to treat the disease. In yet another aspect, the invention provides a method of treating an amyloid-β related disease, comprising administering to a patient a composition comprising a diabody capable of specifically binding to transthyretin and ICAM-1 in an amount effective to treat the disease. In preferred embodiments, the disease is a neurodegenerative disease. The neurodegenerative disease can be any of Alzhiemer's disease, senile sytemic amyloidosis, and familial amyloid polyneuropathy. In other embodiments, the disease can be any amyloid-β related disease, such as familial amyloid cardiomyopathy. In preferred embodiments, the composition is administered to the patient outside of the blood-brain barrier.

One of ordinary skill in the art will understand appropriate dosing regimens for the claimed methods. The present compositions can be administered in any therapeutically effective dosage and on any appropriate schedule. The compositions can suitably be administered to the patient at one time or over a series of treatments. For example, the compositions can be administered daily, semi-weekly, weekly, bi-weekly, semi-monthly, monthly, bi-monthly, semi-annually, or annually. The compositions can be administered once, twice, three times, four times, five times, or more than five times in a day, week, or month. Treatment can continue for one, two, three, four, five, six, seven, eight, nine, ten, eleven, twelve, or more than twelve months as determined by the patient's tolerance and response to the compositions.

For the prevention or treatment of disease, the appropriate dosage of TTR coupled to the ICAM-1-targeting agent or the diabody will depend on the type of disease to be treated, as defined above, the severity and course of the disease, previous therapy, the patient's clinical history and response to the composition, and the discretion of the attending physician. Depending on the type and severity of the disease, about 1 ng/kg to 100 mg/kg of the composition is an initial candidate dosage for administration to the patient, whether, for example, by one or more separate administrations, or by continuous infusion. A typical daily dosage might range from about 1 μg/kg to 100 mg/kg or more, depending on the factors mentioned above. In some embodiments, the contemplated dosage achieves a concentration of TTR between 1 μM and 10 μM in a mammal. For repeated administrations over several days or longer, depending on the condition, the treatment is sustained until a desired therapeutic effect occurs. Such doses can optionally be administered intermittently, e.g. every week or every three weeks (e.g., such that the patient receives from about two to about twenty doses of the therapeutic composition). An initial higher loading dose, followed by one or more lower doses can be administered. The progress of this therapy is easily monitored by conventional techniques and assays. Furthermore, if the compositions are formulated using a carrier for the TTR and ICAM-1-targeting agent as described above, the effective amount of the therapeutic composition delivered can be varied by ratio of TTR:ICAM-1 targeting agent as well as by the dosage to mass ratio.

The compositions of the present invention comprise TTR and at least one ICAM-1-targeting agent and a pharmaceutically acceptable excipient. The composition can be formulated for administration by a route selected from the group consisting of intravenous, intraarterial, intramuscular, intraperitoneal, intrathecal, epidural, topical, percutaneous, subcutaneous, transmucosal, intranasal, or oral. The composition also can comprise additional components such as diluents, adjuvants, excipients, preservatives, and pH adjusting agents, and the like.

Formulations suitable for injectable administration include aqueous and nonaqueous, isotonic sterile injection solutions, which can contain anti-oxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, or tablets.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The active ingredients can also be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are known in the art, e.g., as disclosed in Remington's Pharmaceutical Sciences 16th edition, Osol, A. Ed. (1980). Specifically, liposomes containing the TTR-ICAM-1-targeting agent complex can be prepared by such methods as described in, for example, Rezler et al., J. Am. Chem. Soc. 129(16): 4961-72 (2007); Samad et al., Curr. Drug Deliv. 4(4): 297-305 (2007); and U.S. Pat. Nos. 4,485,045 and 4,544,545. Liposomes with enhanced circulation time are disclosed in, for example, U.S. Pat. No. 5,013,556.

Particularly useful liposomes can be generated by, for example, the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Polypeptides of the present invention can be conjugated to the liposomes as described in, for example, Werle et al., Int. J. Pharm. 370(1-2): 26-32 (2009).

In other embodiments, the active ingredients can be delivered using a natural virus or virus-like particle, a dendrimer, carbon nanoassembly, a polymer carrier, a paramagnetic particle, a ferromagnetic particle, a polymersome, a filomicelle, a micelle or a lipoprotein.

Administration into the airways can provide either systemic or local administration, for example to the trachea and/or the lungs. Such administration can be made via inhalation or via physical application, using aerosols, solutions, and devices such as a bronchoscope. For inhalation, the compositions herein are conveniently delivered from an insufflator, a nebulizer, a pump, a pressurized pack, or other convenient means of delivering an aerosol, non-aerosol spray of a powder, or non-aerosol spray of a liquid. Pressurized packs can comprise a suitable propellant such a liquefied gas or a compressed gas. Liquefied gases include, for example, fluorinated chlorinated hydrocarbons, hydrochlorofluorocarbons, hydrochlorocarbons, hydrocarbons, and hydrocarbon ethers. Compressed gases include, for example, nitrogen, nitrous oxide, and carbon dioxide. In particular, the use of dichlorodifluoromethane, trichlorofluoromethane, dichlorotetrafluoroethane, carbon dioxide or other suitable gas is contemplated. In the case of a pressurized aerosol, the dosage unit can be determined by providing a valve to deliver a controlled amount. In administering a dry powder composition, the powder mix can include a suitable powder base such as lactose or starch. The powder composition can be presented in unit dosage form such as, for example, capsules, cartridges, or blister packs from which the powder can be administered with the aid of an inhalator or insufflator.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, inhaled aerosols, suppositories, mouthwashes, rapidly dissolving tablets, or lozenges. For transdermal administration, the active compounds are formulated into ointments, salves, gels, foams, or creams as generally known in the art.

The pharmaceutical compositions can be delivered using drug delivery systems. Such delivery systems include hyaluronic acid solutions or suspensions of collagen fragments. The drugs can be formulated in microcapsules, designed with appropriate polymeric materials for controlled release, such as polylactic acid, ethylhydroxycellulose, polycaprolactone, polycaprolactone diol, polylysine, polyglycolic, polymaleic acid, poly[N-(2-hydroxypropyl)methylacrylamide] and the like. Particular formulations using drug delivery systems can be in the form of liquid suspensions, ointments, complexes to a bandage, collagen shield or the like.

The invention also provides recombinant DNA or RNA molecules containing a nucleic acid encoding a chimeric protein comprising TTR coupled to an ICAM-1-targeting agent, or a fragment thereof, including but not limited to phages, plasmids, phagemids, cosmids, YACs, BACs, as well as various viral and non-viral vectors well known in the art, and cells transformed or transfected with such recombinant DNA or RNA molecules. Methods for generating such molecules are well known (see, for example, Sambrook et al., The Condensed Protocols From Molecular Cloning: A Laboratory Manual, Cold Spring Harbor, N.Y.: Cold Spring Harbor Laboratory Press (2006)).

The invention further provides a host-vector system comprising a recombinant DNA molecule containing polynucleotide encoding TTR coupled to an ICAM-1-targeting agent, or a fragment thereof within a suitable prokaryotic or eukaryotic host cell, i.e., a cell transformed to express a chimeric protein comprising TTR and an ICAM-1 targeting agent. Examples of suitable eukaryotic host cells include a yeast cell, a plant cell, or an animal cell, such as a mammalian cell or an insect cell (e.g., an S2 cell which allows for expression under metallothionein promoter upon induction by copper sulfate). Examples of suitable mammalian cells include mammalian cells routinely used for the expression of recombinant proteins (e.g., COS, CHO, 293, 293T cells). More particularly, a polynucleotide comprising the coding sequence of TTR coupled to an ICAM-1-targeting agent or a fragment, or an analog or homolog thereof can be used to generate such proteins or fragments thereof using any number of host-vector systems routinely used and widely known in the art.

A wide range of host-vector systems suitable for the expression of the proteins of the present invention are available (see for example, Sambrook et al., 2006, supra), but can employ any vector encoding a chimeric protein comprising transthyretin and an ICAM-1 targeting agent. Preferred vectors for expression include but are not limited to pMT/BiP/V5-His plasmid, which can be expressed in hosts such as E. coli or S2 insect cells.

As discussed herein, redundancy in the genetic code permits variation in encoded gene sequences. In particular, it is known in the art that specific host species often have specific codon preferences, and thus one can adapt the disclosed sequence as preferred for a desired host. For example, preferred analog codon sequences typically have rare codons (i.e., codons having a usage frequency of less than about 20% in known sequences of the desired host) replaced with higher frequency codons. Codon preferences for a specific species are calculated, for example, by utilizing codon usage tables routine to one of ordinary skill in the art.

Additional sequence modifications are known to enhance protein expression in a cellular host. These include elimination of sequences encoding spurious polyadenylation signals, exon/intron splice site signals, transposon-like repeats, and/or other such well-characterized sequences that are deleterious to gene expression. The GC content of the sequence is adjusted to levels average for a given cellular host, as calculated by reference to known genes expressed in the host cell. Where possible, the sequence is modified to avoid predicted hairpin secondary mRNA structures. Other useful modifications include the addition of a translational initiation consensus sequence at the start of the open reading frame, as described in Kozak, Mol. Cell Biol., 9:5073-5080 (1989). One of ordinary skill in the art will understand that the general rule that eukaryotic ribosomes initiate translation exclusively at the 5′ proximal AUG codon is abrogated only under rare conditions (see, e.g., Kozak PNAS 92(7): 2662-2666, (1995) and Kozak, NAR 15(20): 8125-8148 (1987)).

The following examples further illustrate the invention but, of course, should not be construed as in any way limiting its scope.

### Example 1

This example demonstrates the ability of ICAM-1-targeted nanocarriers to target and achieve endocytosis in human brain microvascular endothelial cells (BMVEC).

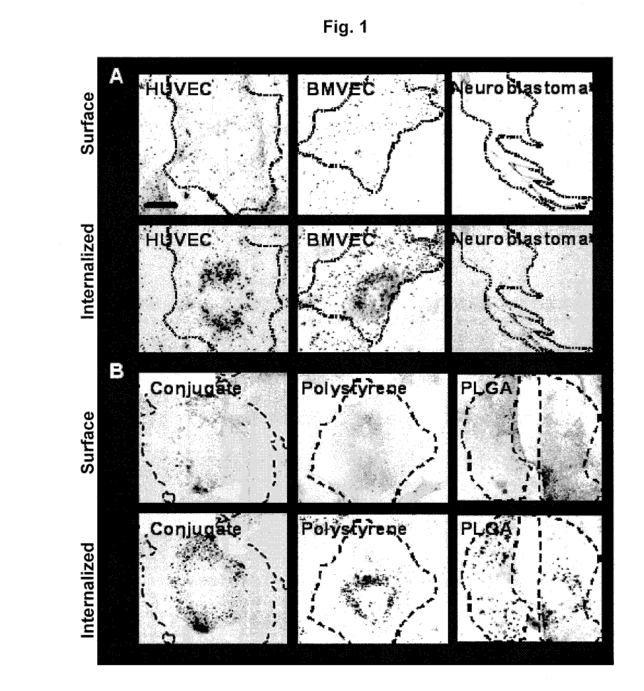

Using TNFα activated BMVEC, human neuroblastoma SH-SY5Y cells, and TNFα-activated human umbilical vein endothelial cells (HUVEC) as a positive control, cells were first incubated for 1 h at 37° C. with 100 nm FITC-labeled (green) anti-ICAM polystyrene carriers. Cells were then washed to remove non-bound carriers, fixed, and stained with texas red-labeled secondary antibody to only detect anti-ICAM carriers accessible in the cell surface. Analysis by fluorescence microscopy showed cell surface-bound carriers in red+green double labeled color in contrast to internalized materials which appeared as single labeled in the green channel. As shown in FIG. 1A, surface binding and internalized endocytic transport of ICAM-1-targeted carriers were detected in both BMVEC and neuroblastoma cells.

Subsequently, TNFα activated HUVEC were incubated for 1 h at 37° C. with anti-ICAM conjugates prepared by coupling via streptavidin biotinylated anti-ICAM, prototype anti-ICAM polystyrene carriers, or biodegradable anti-ICAM poly-lactic co-glycolic acid (PLGA) carriers. Fluorescence microscopy showed that ICAM-1 mediated surface binding and endocytosis were equally efficient for each of these ICAM-1-targeting systems, as shown in FIG. 1B.

These results confirm that ICAM-1 can successfully be targeted and exploited for endocytosis of nanocarriers in a cell culture model.

### Example 2

This example demonstrates the ability of ICAM-1-targeted carriers to reach the brain when injected intravenously in the systemic circulation of laboratory mice.

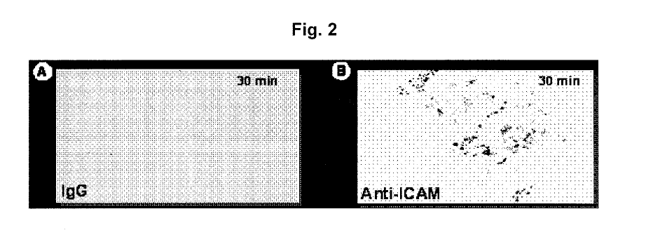

One hundred nanometer diameter, FITC-labeled polystyrene carriers were coated with either IgG (negative control) or anti-ICAM monoclonal antibody and injected intravenously in anesthetized C57B1/6 mice. Thirty minutes after injection, animals were euthanized under anesthesia and the brains were collected and analyzed by fluorescence microscopy to image specific accumulation by ICAM-1-targeting. ICAM-1-targeted carriers were found to accumulate in the brains of the mice, while control carriers coated with non-specific IgG did not (FIGS. 2A-B).

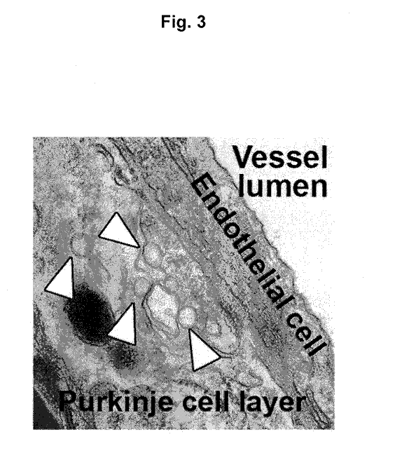

Separately, 100 nm diameter, FITC-labeled polystyrene carriers were coated with anti-ICAM monoclonal antibody and injected intravenously in anesthetized C57B1/6 mice. Thirty minutes, three hours, or eight hours after injection, animals were euthanized under anesthesia and subjected to intracardial perfusion to remove the blood and fix the tissues, and the brain was collected and analyzed by fluorescence microscopy and transmission electron microscopy. At thirty minutes, carriers were observed to bind uniformly to the endothelial walls of brain blood vessels. At three hours, carriers were found to be internalized within endocytic vesicles in endothelial cells in brain vessels (perinuclear “ring-like” localization). At eight hours, transmission electron microscopy showed that carriers had been further transported across the blood-brain barriers into the Purkinje neuronal region of the cerebellum (FIG. 3).

These results show that anti-ICAM-1 carriers successfully target brain endothelial cells in animal models and subsequently traverse the BBB.

### Example 3

This example provides quantitative results for brain targeting of ICAM-1, as compared to the known insulin receptor (InsR) and in two different routes of intravenous administration.

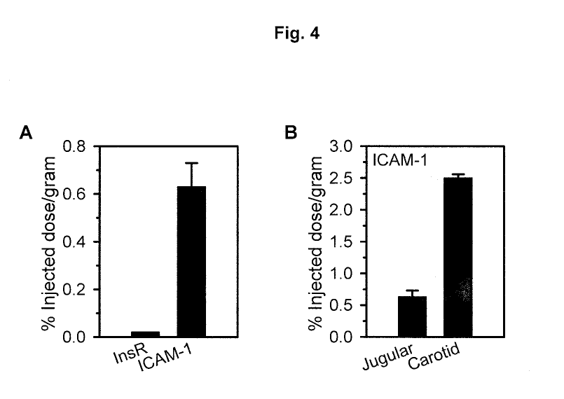

One hundred nanometer diameter polystyrene carriers were coated with anti-ICAM and ¹²⁵I-IgG mixed at 95:5 molar ratio. The resulting carriers were injected intravenously in anesthetized C57B1/6 mice. Thirty minutes after injection, animals were euthanized under anesthesia and the brain was collected to determine the accumulation of carriers, calculated as the percent of the injected dose per gram of the organ. In FIG. 4A, the anti-ICAM data is compared to data reported in Boado et al., Biotechnol. Bioeng. 99:475-84 (2008) for InsR targeting, which has been extensively used for brain delivery of therapeutic compounds.

Subsequently, using the same anti-ICAM carriers (bearing ¹²⁵I-IgG as a tracer), animals were injected either via jugular vein or carotid artery. Brain accumulation of anti-ICAM carriers injected via carotid artery was at least four-fold greater than in animals receiving administration via jugular vein (FIG. 4B).

These results show that carriers coated with anti-ICAM antibody accumulate in the brains of mice to a greater extent than antibodies to receptors previously used for delivery of therapeutics to the brain, and that although jugular and carotid administration both lead to accumulation of anti-ICAM-1 in the brain, administration through a first-pass route to the brain, i.e., carotid artery injection leads to greater accumulation of anti-ICAM-1 than administration through an indirect route, i.e., jugular administration.

### Example 4

This example demonstrates use of an anti-ICAM monoclonal antibody as compared to a 17-mer peptide termed γ3, derived from fibrinogen as described in Altieri et al., J. Biol. Chem. 270:696-9 (1995), an abundant protein in the blood which has affinity domains to several endothelial surface molecules, including ICAM-1.

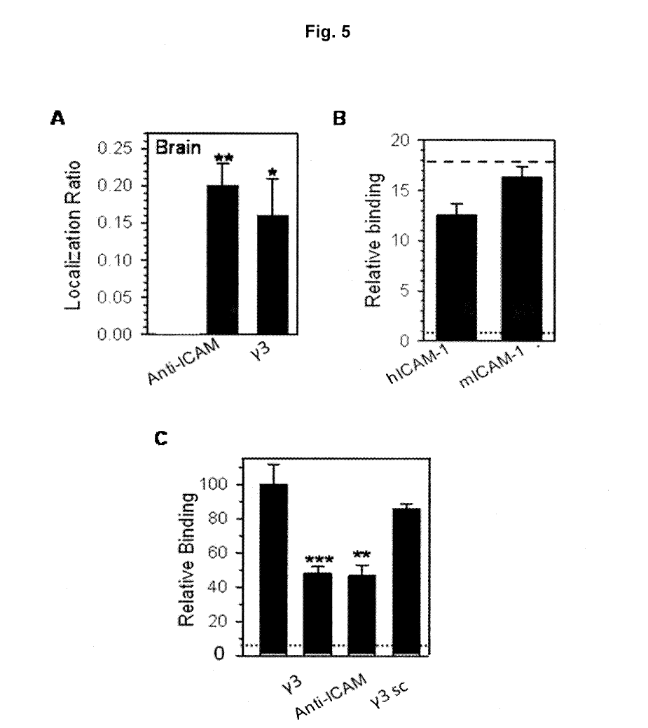

One hundred nanometer diameter polystyrene carriers were coated with either anti-ICAM monoclonal antibody or ICAM-1 affinity peptide γ3 (in both cases carriers contained ¹²⁵I-IgG as a tracer). The resulting carriers were injected intravenously in anesthetized C57B1/6 mice. Thirty minutes after injection, animals were euthanized under anesthesia and the brain was collected to determine the accumulation of carriers. Results were calculated as the Localization Ratio, which represents the percent injected dose/gram in brain divided by percent injected dose/gram in circulation in the blood. As shown in FIG. 5A, brain accumulation was similarly efficient for anti-ICAM and γ3.

FITC-labeled γ3 polystyrene carriers were incubated for 15 min on ELISA versus human or mouse ICAM-1. The membranes were washed and analyzed by fluorescence microscopy to quantify the number of beads bound per area. Non-specific binding of γ3 particles to albumin controls is expressed, for comparison, in FIG. 5B as a dotted line. γ3 carriers target both human and mouse ICAM-1 in a specific manner and with high efficiency, similar to that of anti-ICAM (depicted in FIG. 5B as a dashed line).

Binding of FITC-labeled γ3 carriers to activated HUVEC cells (bars) vs ICAM-1-negative 293 cells (dotted line) was quantified by fluorescence microscopy after 1 h incubation at 37° C., in the absence or presence of excess γ3, anti-ICAM, or γ3 scramble peptide. The γ3 carriers bound to native ICAM-1 expressed by both activated human and mouse endothelial cells, but not control 293 cells which are known to be voided of ICAM-1 expression (FIG. 5C). Targeting to cells was similarly suppressed by excess of free γ3 peptide or anti-ICAM in the media, but not by a peptide with a scrambled γ3 sequence.

These results show that γ3 can be used for brain targeting in mouse and human models and settings.

### Example 5

This example demonstrates that ICAM-1 targeted carriers bearing transthyretin (TTR) on their surface accumulate in the brain at a rate comparable to ICAM-1 targeted carriers with no therapeutic cargo.

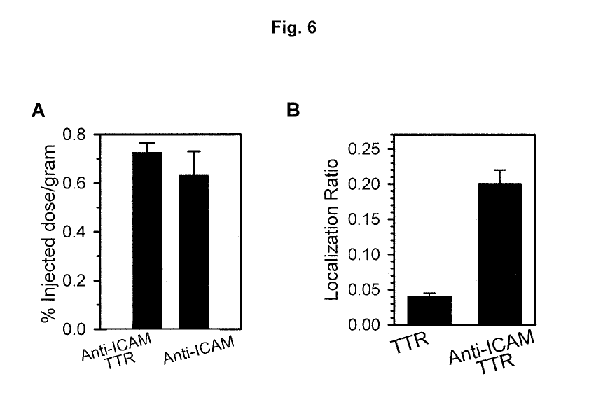

One hundred nanometer diameter polystyrene carriers were coated with anti-ICAM and ¹²⁵I-TTR mixed at 80:20 molar ratio or anti-ICAM and ¹²⁵I-IgG mixed at 95:5 molar ratio. The resulting carriers were injected intravenously in anesthetized C57B1/6 mice. Thirty minutes after injection, animals were euthanized under anesthesia and the brain was collected to determine the accumulation of carriers. Brain accumulation of anti-ICAM/¹²⁵I-IgG and anti-ICAM/¹²⁵I-TTR polystyrene carriers were calculated as percentage of injected dose per gram of brain (FIG. 6A). Brain accumulation of anti-ICAM/¹²⁵I-TTR polystyrene carriers compared to control ¹²⁵I-TTR were calculated as the Localization Ratio (percentage injected dose per gram of brain divided by percentage of injected dose per gram in blood) (FIG. 6B). Delivery of TTR to the brain by ICAM-1-targeted carriers was enhanced by about 5 fold with respect to non-targeted free TTR injected in circulation.

These results show that TTR can be provided in a form capable of targeting the brain.

### Example 6

This example demonstrates the direct coupling of TTR to an ICAM-1 targeting system.

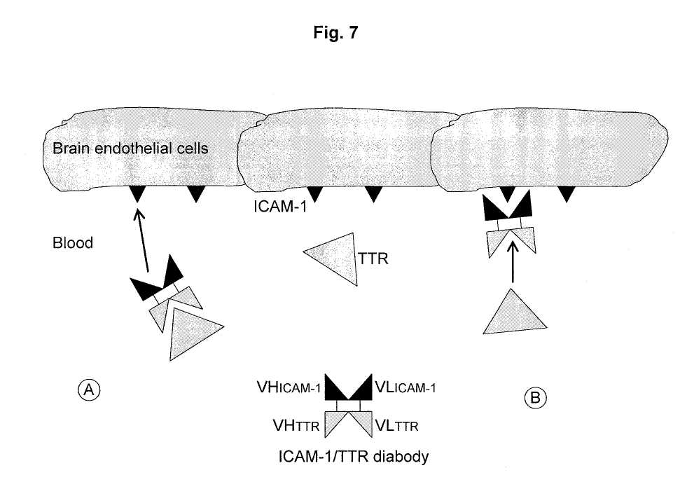

A bispecific antibody, also called a diabody, recognizing both ICAM-1 and TTR (ICAM-1/TTR diabody), is prepared using methods known to one of ordinary skill in the art. The bispecific antibody is injected intravenously in a human or non-human subject. This diabody can then either (A) bind first to TTR in circulation and then the diabody-TTR complex can bind to ICAM-1 expressed in endothelial cells, and/or (B) bind first to ICAM-1 on the endothelial surface and then capture circulating TTR. A schematic of these strategies is provided in FIG. 7.

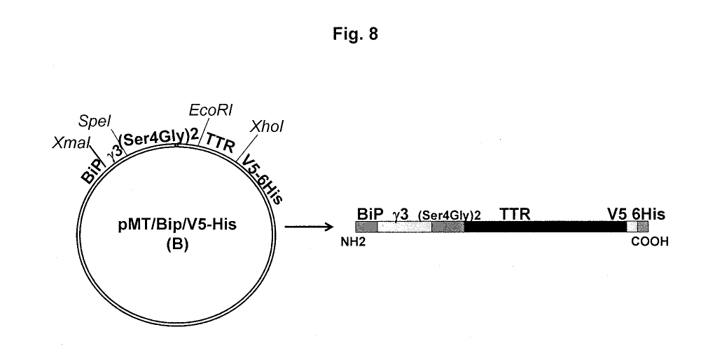

Alternatively, a chimeric TTR protein can be produced from a plasmid as shown in FIG. 8 containing the γ3 sequence (SEQ ID NO:4) cloned at the amino-terminus of the TTR sequence, separated by a (Ser4-Gly)2 peptide spacer to allow independent folding of the targeting and therapeutic moieties of the chimera. The coding sequence for the ICAM-1-targeting peptide γ3 can be formed by hybridization of the forward -F- and reverse -R-oligonucleotides XmaI-γ3-SpeI (SEQ ID NO:1 and SEQ ID NO:2, respectively). The coding sequence for spacer between these peptides and TTR can be formed by hybridization of the forward -F- and reverse -R-oligonucleotides SpeI-(Ser4Gly)2-EcoRI (SEQ ID NO:3 and SEQ ID NO:4, respectively). TTR coding sequence can be amplified by PCR from a plasmid containing TTR cDNA, using the forward primer EcoRI-TTR (SEQ ID NO:5) and reverse primer TTR-XhoI (SEQ ID NO:6). This cassette (generically termed ICAM-1-TTR) can be cloned in a commercial pMT/BiP/V5-His plasmid for amplification under ampicillin selection in E. coli and expression in S2 insect eukaryotic cells using single-site restriction enzyme digestion with XmaI, SpeI, EcoRI, and XhoI, respectively, and subsequent ligation (ICAM-1-TTR plasmid). This cassette allows for expression under metallothionein promoter upon induction by copper sulfate, traffic of the chimeric TTR through the secretory pathway due to presence of BiP, and extracellular secretion after BiP cleavage by S2 cells. The chimeric protein contains a V5 sequence and 6H is tag fused to the carboxyl terminus, and can be purified using a Ni-chelating resin. The resulting protein can be separated by SDS-PAGE and blotted with anti-V5 to trace the V5-tag. Possible modifications of this design include elimination of the BiP, V5 and/or His-tag sequences, elimination of change of the linker, cloning of the targeting peptide in the carboxyl-terminus of TTR, cloning of the targeting peptide both at the amino- and carboxyl-terminus of TTR, introduction of an additional coding sequence for another therapeutic protein and/or targeting peptide, tandem repeats of the targeting peptide to allow for multivalency of ICAM-1 targeting, inclusion of interacting peptides or sequences to promote dimerization, tetramerization, or formation of oligomers of the peptides or the chimera, also to provide multivalent targeting to ICAM-1, cloning into other vectors for expression under different selection markers, in different cell types, in bacteria, by viruses, for protein or gene therapy, among other modifications.

These ICAM-1-targeting strategies provide methods for preparing ICAM-1 targeted TTR for delivery of TTR to the brain which can be used in human and mouse settings, and in cell cultures and animal models.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising transthyretin and an ICAM-1 targeting agent, wherein the transthyretin and ICAM-1 targeting agent are coupled together.

2. The composition of claim 1, wherein the transthyretin and ICAM-1 targeting agent are coupled as a fusion protein.

3. The composition of claim 2, wherein the transthyretin and ICAM-1 targeting agent are coupled as a chimeric protein having a sequence selected from SEQ ID NO: 10 and SEQ ID NO: 13.

4. The composition of claim 1, further comprising a carrier, wherein the transthyretin and ICAM-1 targeting agent are coupled by the carrier.

5. The composition of claim 1, wherein the carrier is a poly-lactic-co-glycolic acid (PLGA) co-polymer.

6. The composition of claim 1, wherein the ICAM-1 targeting agent is selected from the group consisting of an anti-ICAM-1 monoclonal antibody and an ICAM-1 ligand.

7. The composition of claim 6, wherein the ICAM-1 ligand is γ3 (SEQ ID NO:7).

8. The composition of claim 1, further comprising one or more pharmaceutically acceptable excipients.

9. A diabody capable of specifically binding to ICAM-1 and transthyretin.

10. A method of treating an amyloid-β related neurodegenerative disease in a patient comprising administering the composition of claim 1 to the patient, wherein the composition is administered to the patient outside of the blood-brain barrier.

11. A method of treating an amyloid-β related neurodegenerative disease in a patient comprising administering a composition comprising the diabody of claim 9 to the patient, wherein the composition is administered to the patient outside of the blood-brain barrier.

12. The method of claim 11, wherein the diabody is administered in combination with transthyretin.

13. The method of claim 10, wherein the neurodegenerative disease is selected from the group consisting of Alzhiemer's disease, senile sytemic amyloidosis, and familial amyloid polyneuropathy.

14. The method of claim 10, wherein the composition is administered by a route selected from the group consisting of intravenous, intramuscular, transmucosal, intrathecal, epidural, intranasal, oral, topical, and pulmonary.

15. A method of producing a chimeric protein comprising transthyretin and an ICAM-1 targeting agent, the method comprising
(a) expressing transthyretin and the ICAM-1 targeting agent in an expression system; and
(b) recovering the expressed chimeric protein.

16. The method of claim 15, wherein the ICAM-1 targeting agent is γ3 (SEQ ID NO:7).

17. A nucleic acid sequence encoding a chimeric protein comprising transthyretin and an ICAM-1 targeting agent.

18. The nucleic acid sequence of claim 17, wherein the ICAM-1 targeting agent is γ3 (SEQ ID NO:7).

19. The nucleic acid sequence of claim 17, comprising a sequence selected from SEQ ID NO: 8 and SEQ ID NO: 11.

20. A cell transformed to express a chimeric protein comprising transthyretin and an ICAM-1 targeting agent.

21. The cell of claim 20, wherein the ICAM-1 targeting agent is γ3 (SEQ ID NO:7).

22. A vector encoding a chimeric protein comprising transthyretin and an ICAM-1 targeting agent.

23. The vector of claim 22, wherein the ICAM-1 targeting agent is γ3 (SEQ ID NO:7).


SEQUENCE LISTING (13 sequences)

SEQ ID NO: 1 - DNA, 57 bp, Artificial Sequence
  Synthetic
ccgggaataatcaaaagattgttaacctgaaagagaaggtagcccagcttgaagcaa

SEQ ID NO: 2 - DNA, 57 bp, Artificial Sequence
  Synthetic
ctagttgcttcaagctgggctaccttctctttcaggttaacaatcttttgattattc

SEQ ID NO: 3 - DNA, 42 bp, Artificial Sequence
  Synthetic
actagttcttcttcttctggctcttcttcttctggcgaattc

SEQ ID NO: 4 - DNA, 42 bp, Artificial Sequence
  Synthetic
gaattcgccagaagaagaagagccagaagaagaagaactagt

SEQ ID NO: 5 - DNA, 24 bp, Artificial Sequence
  Synthetic
aattcgcttctcatcgtctgctcc

SEQ ID NO: 6 - DNA, 26 bp, Artificial Sequence
  Synthetic
tcgagaaggaaccctaaccactgctg

SEQ ID NO: 7 - protein, 17 aa, Artificial Sequence
  Synthetic
NNQKIVNLKEKVAQLEA

SEQ ID NO: 8 - DNA, 1110 bp, Artificial Sequence
  Synthetic
atgaaaatcgaagaaggtaaactgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaagctatctctggcgatagtctgatcagcctggctagcacaggaaaaagagtttctattaaagatttgttagatgaaaaagattttgaaatatgggcaattaatgaacagacgatgaagctagaatcagctaaagttagtcgtgtattttgtactggcaaaaagctagtttatattctaaaaactcgactaggtagaactatcaaggcaacagcaaatcatagatttttaactattgatggttggaaaagattagatgagctatctttaaaagagcatattgctctaccccgtaaactagaaagctcctctttacaattgtcaccagaaatagaaaagttgtctcagagtgatatttactgggactccatcgtttctattacggagactggagtcgaagaggtttttgatttgactgtgccaggaccacataactttgtcgcgaatgacatcattgtacacaacaataatcaaaagatcgttaaccttaaagagaaggtagcccagcttgaagcatcgtcgtcgtcgggctcgtcgtcgtcgggcgcagcaggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 9 - protein, 369 aa, Artificial Sequence
  Synthetic
MKIEEGKLTNPGVSAWQVNTAYTAGQLVTYNGKTYKCLQPHTSLAGWEPSNVPALWQLQAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLSPEIEKLSQSDIYWDSIVSITETGVEEVFDLTVPGPHNFVANDIIVHNNNQKIVNLKEKVAQLEASSSSGSSSSGAAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 10 - protein, 156 aa, Artificial Sequence
  Synthetic
NNQKIVNLKEKVAQLEASSSSGSSSSGAAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 11 - DNA, 1110 bp, Artificial Sequence
  Synthetic
atgaaaatcgaagaaggtaaactgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaagctatctctggcgatagtctgatcagcctggctagcacaggaaaaagagtttctattaaagatttgttagatgaaaaagattttgaaatatgggcaattaatgaacagacgatgaagctagaatcagctaaagttagtcgtgtattttgtactggcaaaaagctagtttatattctaaaaactcgactaggtagaactatcaaggcaacagcaaatcatagatttttaactattgatggttggaaaagattagatgagctatctttaaaagagcatattgctctaccccgtaaactagaaagctcctctttacaattgtcaccagaaatagaaaagttgtctcagagtgatatttactgggactccatcgtttctattacggagactggagtcgaagaggtttttgatttgactgtgccaggaccacataactttgtcgcgaatgacatcattgtacacaacaataatcaaaagcttgttaacatcaaagagaaggtagcccagatcgaagcatcgtcgtcgtcgggctcgtcgtcgtcgggcgcagcaggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 12 - protein, 369 aa, Artificial Sequence [sequence omitted]

SEQ ID NO: 13 - protein, 156 aa, Artificial Sequence
  Synthetic
NNQKLVNIKEKVAQIEASSSSGSSSSGAAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE